(19) United States
(12) Patent Application Publication
(10) Pub. No.: US 2012/0231458 A1
(43) Pub. Date: Sep. 13, 2012

(54) METHOD OF ACQUIRING STANDARD CURVE IN REAL-TIME PCR

(75) Inventor: Soon-min BAE, Changwon-city (KR)

(73) Assignee: SAMSUNG TECHWIN CO., LTD., Changwon-city (KR)

(21) Appl. No.: 13/416,023

(22) Filed: Mar. 9, 2012

(30) Foreign Application Priority Data
Mar. 9, 2011 (KR) 10-2011-0021047

(51) Int. Cl. C12Q 1/68

(52) U.S. Cl. 435/6.11

(57) ABSTRACT

A method of acquiring a standard curve for quantifying polynucleotide is provided. The method includes: (a) performing a real-time polynucleotide chain reaction (PCR) for plural samples having different initial polynucleotide concentrations, the PCR being performed with respect to plural amplification cycle numbers using detectable probes providing a signal according to an amount of polynucleotide; (b) acquiring plural amplification profile curves with respect to signal intensity values provided by the probes according to the amplification cycle numbers; (c) selecting one threshold from among the signal intensity values; (d) calculating amplification cycle numbers corresponding to the selected thresholds from the plural amplification profile curves, and determining the calculated amplification cycle numbers as threshold cycle (Ct) values corresponding to each of the initial polynucleotide concentrations; (e) selecting at least two Ct values among the Ct values determined in (d); and (f) acquiring a standard curve from the selected Ct values.

### CROSS-REFERENCE TO RELATED PATENT APPLICATION

This application claims priority from Korean Patent Application No. 10-2011-0021047 filed on Mar. 9, 2011 in the Korean Intellectual Property Office, the disclosure of which is incorporated herein in its entirety by reference.

### BACKGROUND

1. Field

Methods consistent with exemplary embodiments relate to acquiring a standard curve from an amplification profile curve indicating an amount of polynucleotide according to the cycle number obtained by performing real-time polynucleotide chain reaction (PCR).

2. Description of the Related Art

A real-time polynucleotide chain reaction (PCR) monitors an increase of a PCR amplification product, and thus, polynucleotide may be quantitative. The real-time PCR is widely used in measuring gene expression, monitoring biological reactions against stimulation, genomic-level gene quantification, biological studies such as detection of pathogens, and clinical analysis.

In real-time PCR, an amount of a PCR amplification product may be detected by a fluorescence signal. The detection method may include intercalation using an intercalator which indicates fluorescence when bonded to double helix DNA, or using oligonucleotide, in which the 5′ end and 3′ end are marked as a fluorescent material and a quencher material, respectively. If the above method is used, an intensity of a fluorescence signal increases according to an increased amount of polynucleotide while the real-time PCR progresses, and a user may acquire an amplification profile curve indicating the intensity of the fluorescence signal according to the number of an amplification cycle.

In general, an amplification profile curve is divided into a baseline region indicating a background fluorescence signal, in which a substantial amount of polynucleotide is not reflected, an exponential region indicating an increase in intensity of a fluorescence signal by reflecting an increase in an amount of polynucleotide, and a plateau region, in which an increase in intensity of a fluorescence signal is no longer observed as a PCR reaction saturates (FIG. 1).

The amplification curve has the baseline region in an initial stage of the PCR reaction, because an amount of a PCR product does not reach a detectable amount by a fluorescence signal.

In general, the intensity of a fluorescence signal at a point changing from the baseline region to the exponential region, that is, when an amount of a PCR amplification product reaches an amount detectable by fluorescence, is referred to as a threshold, and a amplification cycle number corresponding to the threshold in the amplification profile curve is referred to as a threshold cycle (Ct). When real-time PCRs are each performed by varying initial concentration of polynucleotide, as an initial amount of polynucleotide increases, the amplification cycle number reaching a detectable amount decreases, and a Ct value decreases (FIG. 2). Accordingly, a log value of the initial amount of polynucleotide is inversely proportional to the Ct value, and a function indicating the Ct value with respect to the log value of the initial amount of polynucleotide is a standard curve (FIG. 3). Once the standard curve is determined, an amount of polynucleotide in an unknown sample can be estimated by substituting its Ct value to the standard curve. Since the standard curve is determined according to the threshold, it is important to determine the threshold in quantification of polynucleotide in the real-time PCR. To determine a more precise threshold value, several methods are used. For example, various methods determine the threshold value as the value where the fluorescence reaches a predetermined signal level called the arbitrary fluorescence value (AFL). Other methods use the amplification cycle number where the second derivative of fluorescence vs. cycle number reaches a maximum. When the AFL is used, the threshold may be significantly affected by a slight change of an average baseline fluorescent level in an initial PCR cycle. When derivative methods are used, an outlier may significantly affect the threshold. Further, in derivative methods, separate analysis needs to be performed on each amplification profile curve, whereas the standard curve is determined from Ct values of all amplification profile curves. When an appropriate standard curve is not formed by using the above methods, in general, a threshold may be directly determined by a user's eyes. In this case, as subjective determination of a user may be made, the accuracy of quantification of polynucleotide may be reduced. Accordingly, for accurate quantification of polynucleotide, an accurate threshold should be determined from an amplification profile curve of the real-time PCR, and thus, an accurate standard curve needs to be acquired.

### SUMMARY

One or more exemplary embodiments provide a method of acquiring a standard curve from an amplification profile curve indicating an amount of polynucleotide according to an amplification cycle number in real-time PCR.

One or more exemplary embodiments also provide a method of accurately quantifying polynucleotide.

According to an aspect of an exemplary embodiment, there is provided a method of acquiring a standard curve for quantifying polynucleotide by performing a real-time polynucleotide chain reaction (PCR), the method including: (a) performing the real-time PCR for a plurality of samples having different initial polynucleotide concentrations, wherein the PCR is performed with respect to a plurality of amplification cycle numbers using detectable probes which provide a signal according to an amount of polynucleotide; (b) acquiring a plurality of amplification profile curves with respect to signal intensity values provided by the probes according to the amplification cycle number; (c) selecting one threshold from among the signal intensity values; (d) calculating amplification cycle numbers corresponding to the selected thresholds from the plurality of amplification profile curves, and determining the calculated amplification cycle numbers as threshold cycle (Ct) values corresponding to each of the initial polynucleotide concentrations; (e) selecting at least two Ct values among the Ct values determined in (d); and (f) acquiring a standard curve from the selected Ct values.

According to the above method, the standard curve for quantifying polynucleotide is acquired by performing a real-time PCR.

The term “real-time PCR” is an improved technology of PCR, which amplifies polynucleotide by using polymerase, and may monitor an increase in an amount of amplified polynucleotide in real-time by intensity of fluorescence which is bonded to polynucleotide. The PCR denotes a reaction that amplifies polynucleotide by repeatedly performing three temperature varying processes including denaturation, annealing, and elongation after placing polynucleotide with dNTP, a primer, and polymerase. In the real-time PCR, intercalation, TaqMan probe, or cycling probe may be used, according to a used fluorescent material, without being limited thereto.

The method above includes (a) performing the real-time PCR for a plurality of samples having different initial polynucleotide concentrations, wherein the PCR includes plural cycle numbers and is performed with respect to a plurality of amplification cycle numbers using detectable probes which provide a signal according to an amount of polynucleotide. The different initial polynucleotide concentrations may be formed by diluting a polynucleotide sample, in which concentration thereof is known, in stages. The detectable probes include an intercalation dye such as SYBR Green I, Ethidium bromide, or YO-PRO-1 BOXTO, fluoregenic hybridization oligoprobe, in which donor fluorephore (FITC) is marked at a 3′ end thereof and acceptor fluorophore is marked at a 5′ end thereof, TaqMan probe, hairpin oligoprobe, or self-fluorescing amplicon (sunrise primer & scorpion primer), without being limited thereto.

The method above includes (b) acquiring a plurality of amplification profile curves with respect to signal intensity values provided by the probes according to the amplification cycle number.

While the real-time PCR is performed, as the cycle number increases, an amount of polynucleotide increases and the signal intensity values provided by the probes increase. The amplification profile curve denotes a relational function indicating the signal intensity values provided by the probes according to the amplification cycle number by detecting the signal intensity values. More specifically, the amplification profile curve may be indicated by a graph, in which Rn (detected signal intensity/signal intensity of passive reference probe) or ΔRn (Rn−Rn of a baseline region) is a y-axis, and the cycle number is an x-axis.

The method above includes (c) selecting one threshold from among the signal intensity values.

The threshold may be a value arbitrarily selected from among the signal intensity values of the amplification profile curve. More specifically, the threshold may be a signal intensity value between the baseline region and the plateau region in the amplification profile curve, for example, a signal intensity value at a spot passing from the baseline region to the exponential region in the amplification profile curve. The baseline region indicates a region where a signal intensity value, at an initial real-time PCR, is not exponentially increased in the amplification profile curve. The exponential region indicates a region where a signal intensity value increases in the amplification profile curve. The plateau region indicates a region where a signal intensity value indicated after the exponential region is not increased in the amplification profile curve. The baseline region is indicated because an amount of polynucleotide does not reach an amount of polynucleotide having a detectable signal intensity value.

The method above includes (d) calculating amplification cycle numbers corresponding to the selected thresholds from the plurality of amplification profile curves, and determining the calculated amplification cycle numbers as threshold cycle (Ct) values corresponding to the each initial polynucleotide concentrations.

The term “threshold cycle” denotes the cycle number when the signal intensity value is the threshold in the amplification profile curve. As an initial amount of polynucleotide increases, the exponential region starts at lower cycle number. Accordingly, as an initial amount of polynucleotide increases, a lower Ct value may be obtained.

The method above includes (e) selecting at least two Ct values among the Ct values determined in (d); and (f) acquiring a standard curve from the selected Ct values.

The term “standard curve” denotes a relational function indicating an initial amount of polynucleotide according to Ct values. The Ct values determined in (d) each correspond to the different initial polynucleotide concentrations. Accordingly, a function indicating the relationship between the Ct values and the initial amount of polynucleotide in (a) may be determined based on the Ct values, and the function may be the standard curve.

In the above method, in (e), at least two Ct values is selected among the determined Ct values in (d), and thus, the standard curve in (f) may be acquired from the at least two selected Ct values. In general, when the standard curve is acquired in a real-time PCR, all Ct values are used. However, a part of Ct values may be used to exclude outlier Ct values.

The standard curve may be acquired as a linear function by performing a linear regression analysis from at least two Ct values that correspond to each different initial amount of polynucleotide.

Also, the standard curve may be represented by a relational function of a log value of the initial amount of polynucleotide according to the Ct values. When the standard curve is once acquired by performing a real-time PCR on polynucleotide samples, in which initial concentrations are known, the real-time PCR is performed on samples, in which an initial amount of polynucleotide is unknown, under the same condition and the same threshold is applied so as to obtain the Ct values. The Ct values are applied to the acquired standard curve so that the initial amount of polynucleotide may be identified.

According to an aspect of another exemplary embodiment, the method of acquiring the standard curve for quantifying polynucleotide by performing real-time PCR including (a), (b), (c), (d), (e), and (f) above may further include: (g) repeating (e) and (f) a plurality of times so that groups including at least two Ct values selected in (e) are different each time when (e) is repeated; and (h) selecting a standard curve, which excludes outliers to the maximum and includes inliers to the maximum from among the Ct values determined in (d), from among the standard curves acquired in (g).

When it is assumed that most acquired data complies with a fixed rule, the term “outlier” denotes noise values which do not comply with the rule from among the acquired data. The noise values may be generated due to an error in measuring equipment, lack of experience of an experimenter in experiment, and contamination of samples.

The outlier used herein refers to Ct values in which a difference between the Ct values determined in (d) and the standard curve is beyond a set range. The Ct values determined in (d) may be present on the standard curve or outside the standard curve. In this regard, if there is present a difference between the Ct values determined in (d) and the standard curve for the same nucleic acid amount and such a difference is beyond a set range, i.e., a set cycle number, the Ct values determined in (d) may be regarded as outliers.

In this regard, the set range may be from about 0.1 to about 1 cycle. For example, the set range may be from about 0.2 to about 0.5 cycles, and for example, 0.25 cycles. The set range may be appropriately adjusted to increase the accuracy of the standard curve.

When it is assumed that most acquired data complies with a fixed rule, the term “inlier” denotes most data that complies with the rule.

The inlier used herein refers to Ct values in which a difference between the Ct values determined in (d) and the standard curve is within a set range. The Ct values determined in (d) may be present on the standard curve or outside the standard curve. In this regard, if there is present a difference between the Ct values determined in (d) and the standard curve for the same nucleic acid amount and such a difference is within a set range, i.e., a set cycle number, the Ct values determined in (d) may be regarded as inliers. For example, the set range may be from about 0.1 to about 1 cycle. For example, the set range may be from about 0.2 to about 0.5 cycles, and for example, 0.25 cycles. The set range may be appropriately adjusted to increase the accuracy of the standard curve.

When outliers are excluded to the maximum and more inliers are used in outputting a general rule, with which the acquired data is complied, a more accurate rule may be output, compared with when both outliers and inliers are used.

In (g), the Ct values selected in (e) are different each time when (e) is repeated, and (e) and (f) are repeated a plurality of times so that the standard curves, which are different from each other, may be acquired by the plurality of times of repeating. In (h), one standard curve is finally selected from among the standard curves by the plurality of times of repeating. In (h), the standard curve, which excludes outliers to the maximum and includes inliers to the maximum, indicates a standard curve which may well predict a tendency of most Ct values determined in (d). The selected standard curve may vary according to a range of the inlier. If the Ct value on the standard curve is defined as an inlier, a standard curve including the most Ct values determined in (d) may be selected. If the Ct value within a fixed error range from the standard curve is defined as an inlier, a standard value including the most Ct values within the fixed error range from the standard curve may be selected. In the latter case, a standard curve having the smallest average in errors between the standard curve and inliers may be selected.

Accordingly, the standard curve is not output by using all Ct values (including outliers) determined by one threshold, and instead, different standard curves are repeatedly output by using a part of Ct values, which are different from each other. Then, the standard curve, which corresponds to inliers to the maximum, is selected so that the standard curve, in which outliers are not reflected, may be acquired. When the frequency of repeatedly outputting the standard curve is all number of cases in which a part of Ct values can be selected, the selected standard curve may be an optimum standard curve in a fixed condition and a fixed threshold.

According to an aspect of another exemplary embodiment, the method of acquiring the standard curve for quantifying polynucleotide by performing real-time PCR including (a), (b), (c), (d), (e), (f), (g), and (h) above may further include: (i) repeating (c), (d), (g), and (h) a plurality of times so that the threshold selected in (c) is different each time when (c) is repeated; and (j) selecting a standard curve, which excludes the outliers and includes the inliers from among the Ct values determined in (d), from among the standard curves acquired in (h).

The threshold selected in (i) may be a value that is arbitrarily selected from among the signal intensity values applied to the amplification profile curve in (b). When (i) is performed, the standard curves including inliers to the maximum are selected with respect to each different threshold, and, in (j), a standard curve including inliers to the maximum is selected from among the standard curves.

In the derivative method which is a general method of outputting a standard curve, a secondary differential coefficient from each amplification profile curve is used to determine thresholds, and thus, the thresholds may be different in each amplification profile curve. Accordingly, it is hard to regard that the standard curve output by selecting one of the thresholds reflects data in all amplification profile curves. According to an aspect of another exemplary embodiment, a standard curve, to which the Ct values output from each amplification profile curve are reflected mostly, is selected, and thus a standard curve, to which tendency of a large number of amplification profile curves are reflected, may be acquired. Also, differentiation is not performed on each amplification profile curve, and instead, Ct values for one threshold are calculated once. Thus, time used for calculation may be reduced.

According to an aspect of another exemplary embodiment, there is provided a method of acquiring a standard curve for quantifying polynucleotide by performing real-time polynucleotide chain reaction (real-time PCR), the method including: (a1) performing the real-time PCR for a plurality of samples having different initial polynucleotide concentrations, wherein the PCR is performed with respect to a plurality of amplification cycle numbers using detectable probes which provide a signal according to an amount of polynucleotide; (b1) acquiring a plurality of amplification profile curves with respect to signal intensity values provided by the probes according to the amplification cycle numbers; (c1) selecting a plurality of different thresholds from among the signal intensity values; (d1) calculating amplification cycle numbers corresponding to the selected thresholds from the plurality of amplification profile curves, and determining the calculated amplification cycle numbers as threshold cycle (Ct) values corresponding to each of the initial polynucleotide concentrations; (e1) acquiring a plurality of standard curves for the plurality of selected thresholds based on the Ct values; and (f1) selecting a standard curve, which excludes Ct values beyond a set range as outliers, and includes Ct values within the set range as inliers, from among the Ct values determined in (d1), from among the plurality of acquired standard curves.

Technical terms described in the previous embodiment are also applied to the current embodiment. According to the current embodiment, a plurality of thresholds are selected to acquire a standard curve for each threshold, and then, the standard curve including the most inliers is selected. Accordingly, an optimum standard curve including the most inliers may be selected from among the standard curves according to the selected thresholds.

In (e1), at least two of the plurality of Ct values determined in (d1) is selected, and thus, the standard curves may be acquired from the at least two selected Ct values.

In (e1), two of the Ct values determined in (d1) are selected to acquire a standard curve. When the standard curve according to the selected threshold is output, the standard curve is not output by using all Ct values, and instead, the standard curve is output by using a part of the Ct values to exclude influences of outliers in outputting the standard curve.

According to an aspect of another exemplary embodiment, a standard curve having the smallest average value of errors between the acquired standard curves and all Ct values, which is determined in (d) or (d1) of the methods in the above exemplary embodiments using thresholds corresponding to the acquired standard curves, can be selected in (h), (j), and (f1). The all Ct values, which is determined in (d) or (d1) using thresholds corresponding to the acquired standard curves, denote all Ct values determined in (d) or (d1) with the same thresholds as thresholds used to acquire the standard curve. The error denotes a difference between the acquired standard curve and an observed Ct values for the same initial polynucleotide concentration.

According to an aspect of another exemplary embodiment, the average value of errors may be calculated excluding an error having a fixed value or above. A Ct value having a significantly large error with the standard curve may be an outlier, and thus, calculating the average value of errors without said outlier will be helpful to select an accurate standard curve. The fixed value denotes a value by which Ct values having an error above the fixed value are determined as the outlier, and may be arbitrarily set by a user. More specifically, the fixed value may be about 0.01 to 1 Ct values, more preferably, about 0.1 to about 0.7 Ct values, for example, about 0.2 to about 0.6 Ct values, for example, about 0.4 to about 0.5 Ct values, and for example, 0.25 Ct values without being limited thereto. According to an aspect of another exemplary embodiment, the standard curve having the most corresponding Ct values on the standard curve is selected from among the plurality of acquired standard curves in (h), (j), and (f1). The corresponding Ct values denote all Ct values determined in (d) or (d1) with the same threshold as the threshold used to acquire the standard curve. The corresponding Ct values being on the standard curve denote that the corresponding Ct values are included in the standard curve.

According to an aspect of another exemplary embodiment, the number of the Ct values selected in (e) may be two. When the number of the Ct values determined in (d) is two, a standard curve, which is a linear line, may be output. The number of the Ct values selected in (e) may be in the range of between two and the number of all Ct values. When the number of selected Ct values is three or more, a linear regression analysis may be used to output a standard curve, which is a linear line, without being limited thereto.

According to an aspect of another exemplary embodiment, (e) and (f) may be repeated by a plurality of times so that different groups can be selected in (g), that is, the groups including at least two Ct values selected in (e) are different each time when (e) is repeated. For example, in (e), when n Ct values are selected and the number of all Ct values determined in (d) is p, (e) and (f) are repeated by ₚCₙ times. ₚCₙ indicates a combination symbol used in probability and statistics, and denotes the number of all methods of selecting n, which is different from each other, from among p variables(_{P}Cₙ=p!/n!(p−n)!). The standard curves are acquired by the number of all cases, and the standard curve including the most inliers is selected from among the standard curves. Then, an optimum standard curve can be acquired for the given thresholds and condition.

According to an aspect of another exemplary embodiment, fixed sections in the signal intensity values may be determined, and values that equally divide the sections may be selected as the thresholds in (c) and (c1) of the methods in the above exemplary embodiments. When arbitrary two values are selected from among the signal intensity values of the amplification profile curve, the fixed section denotes a section between the two values. The two values that determine the fixed sections may be arbitrarily selected by a user. However, in general, a spot passing from the baseline region to the exponential region is known as a threshold for accurate quantification. Thus, the fixed sections may be determined to include signal intensity values around the spot passing from the baseline region to the exponential region on the amplification profile curve. The values that equally divide the fixed sections denote values that divide the fixed sections by a fixed range. For example, when the fixed section corresponds to 1 through 5 and is equally divided with a range of 1, values thereof are 1, 2, 3, 4, and 5. The thresholds are selected by equally dividing the fixed section because an accuracy of the thresholds may be examined throughout the selected fixed sections without a difference in each divided section. Preferably, but not necessarily, a part, which is predicted that a threshold for accurate quantification exists (around the spot passing from the baseline region to the exponential region), can be divided densely and equally, and divided values can be thresholds. Then, a threshold that accurately outputs the standard curve can be selected.

According to an aspect of another exemplary embodiment, a method of acquiring a threshold for quantifying polynucleotide by performing a real-time PCR includes:

(a) performing the real-time PCR for a plurality of samples having different initial polynucleotide concentrations, wherein the PCR is performed with respect to a plurality of amplification cycle numbers using detectable probes which provide a signal according to an amount of polynucleotide;

(b) acquiring a plurality of amplification profile curves with respect to signal intensity values provided by the probes according to the amplification cycle numbers;

(c) selecting one threshold from among the signal intensity values;

(d) calculating amplification cycle numbers corresponding to the selected thresholds from the plurality of amplification profile curves, and determining the calculated amplification cycle numbers as threshold cycle (Ct) values corresponding to each of the initial polynucleotide concentrations;

(e) selecting at least two Ct values among the Ct values determined in (d); and

(f) acquiring a standard curve from the selected Ct values.

(g) repeating (e) and (f) a plurality of times so that groups including at least two Ct values selected in (e) are different each time when (e) is repeated; and

(h) selecting a standard curve, which excludes outliers to the maximum and includes inliers to the maximum from among the Ct values determined in (d), from among the standard curves acquired in (g)

(i) repeating (c), (d), (g), and (h) a plurality of times so that the threshold selected in (c) is different each time when (c) is repeated; and

(j) selecting a standard curve, which excludes the outliers to the maximum and includes the inliers to the maximum from among the Ct values determined in (d), from among the standard curves acquired in (h)

(k) selecting the threshold based on the selected standard curve determined in (j).

The definition of terminologies described in exemplary embodiments of the method of acquiring a standard curve is the same as that in exemplary embodiments for selecting a threshold.

According to an aspect of another exemplary embodiment, (h) may be replaced by a process of selecting a standard curve, which includes inliers to the maximum among the Ct values determined in (d), from among the standard curves acquired in (f).

The outliers used herein refer to Ct values in which a difference between the Ct values determined in (d) and the standard curve is beyond a set range. For example, the set range may be from about 0.1 to about 1 cycle. For example, the set range may be from about 0.2 to about 0.5 cycles, and for example, 0.25 cycles. The set range may be appropriately adjusted to increase the accuracy of the standard curve.

The inliers used herein refers to Ct values in which a difference between the Ct values determined in (d) and the standard curve is within a set range. For example, the set range may be from about 0.1 to about 1 cycle. For example, the set range may be from about 0.1 to about 0.7 cycles, for example, about 0.2 to about 0.6 cycles, and for example, 0.25 cycles. The set range may be appropriately adjusted to increase the accuracy of the standard curve.

According to an aspect of another exemplary embodiment, (h) may be replaced by a process of selecting a standard curve having the smallest error between the standard curve acquired in (0 and the Ct values determined in (d).

The error indicates a difference between the Ct values determined in (d) and the standard curve acquired in (g) for the same nucleic acid amount. In other words, the error denotes a difference between the acquired standard curve and observed Ct values for the same initial polynucleotide concentration.

The method may further include selecting a threshold based on the standard curve selected in (h).

Since an optimal standard curve is selected in (h), an optimal threshold for a certain concentration of nucleic acid may be calculated using the standard curve. In addition, the amount of nucleic acid that has a certain threshold and an unknown concentration may be accurately calculated.

According to an aspect of another exemplary embodiment, a method of acquiring a threshold for quantifying polynucleotide by performing a real-time polynucleotide chain reaction (PCR) includes:

(a1) performing the real-time PCR for a plurality of samples having different initial polynucleotide concentrations, wherein the PCR is performed with respect to a plurality of amplification cycle numbers using detectable probes which provide a signal according to an amount of polynucleotide;

(b1) acquiring a plurality of amplification profile curves with respect to signal intensity values provided by the probes according to the amplification cycle numbers;

(c1) selecting a plurality of different thresholds from among the signal intensity values;

(d1) calculating amplification cycle numbers corresponding to the selected thresholds from the plurality of amplification profile curves, and determining the calculated amplification cycle numbers as threshold cycle (Ct) values corresponding to each of the initial polynucleotide concentrations;

(e1) acquiring a plurality of standard curves for the plurality of selected thresholds based on the Ct values; and

(f1) selecting a standard curve, which excludes Ct values beyond a set range as outliers, and includes Ct values within the set range as inliers, from among the Ct values determined in (d1), from among the plurality of acquired standard curves.

(g1) selecting the threshold based on the selected standard curve determined in (f1)

The definition of terminologies described in exemplary embodiments of the method of acquiring a standard curve is the same as that in exemplary embodiments for selecting a threshold.

According to an aspect of another exemplary embodiment, (g1) may be replaced by a process of selecting a standard curve including most inliers among the Ct values determined in (d1), among the standard curves acquired in (f1).

The outliers used herein refer to Ct values in which a difference between the Ct values determined in (d1) and the standard curve is beyond a set range. For example, the set range may be from about 0.1 to about 1 cycle. For example, the set range may be from about 0.2 to about 0.5 cycles, and for example, 0.25 cycles. The set range may be appropriately adjusted to increase the accuracy of the standard curve.

The inliers used herein refers to Ct values in which a difference between the Ct values determined in (d1) and the standard curve is within a set range. For example, the set range may be from about 0.01 to about 1 cycle. For example, the set range may be from about 0.2 to about 0.5 cycles, and for example, 0.25 cycles. The set range may be appropriately adjusted to increase the accuracy of the standard curve.

According to an aspect of another exemplary embodiment, (g1) may be replaced by a process of selecting a standard curve having the smallest error between the standard curve acquired in (f1) and the Ct values determined in (d1).

The error indicates a difference between the Ct values determined in (d1) and the standard curve acquired in (f1) for the same nucleic acid amount. In other words, the error denotes a difference between the acquired standard curve and observed Ct values for the same initial polynucleotide concentration.

The method may further include selecting a threshold based on the standard curve selected in (g1).

Since an optimal standard curve is selected in (g1), an optimal threshold for a certain concentration of nucleic acid may be calculated using the standard curve. In addition, the amount of nucleic acid that has a certain threshold and an unknown concentration may be accurately calculated.

### BRIEF DESCRIPTION OF THE DRAWINGS

The above and other aspects will become more apparent by describing in detail exemplary embodiments with reference to the attached drawings, in which:

FIG. 1 is a general amplification profile curve acquired by performing a real-time polynucleotide chain reaction (PCR);

FIG. 2 illustrates amplification profile curves, thresholds, and threshold cycle (Ct) values when the real-time PCR is performed with varying an initial amount of polynucleotide;

FIG. 3 illustrates an example of a standard curve determined using Ct values acquired from FIG. 2;

FIG. 4 illustrates amplification profile curves acquired by performing real-time PCR according to Example 1; and

FIGS. 5A and 5B illustrate, based on the same data, a standard curve (FIG. 5A) acquired according to an exemplary embodiment and a standard curve (FIG. 5B) acquired by using an Applied Biosystems 7500 Real-Time PCR apparatus.

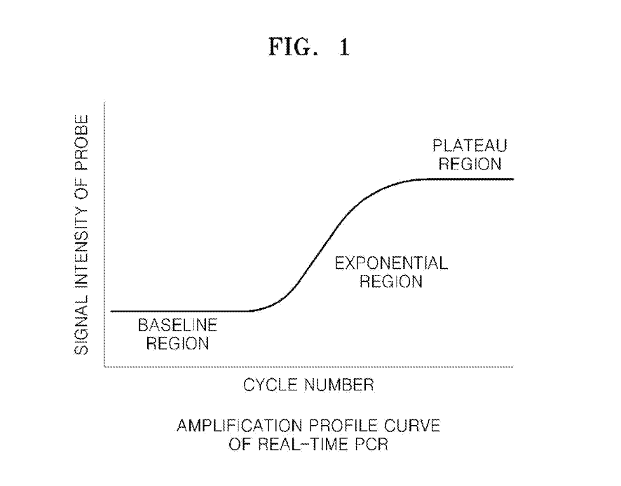

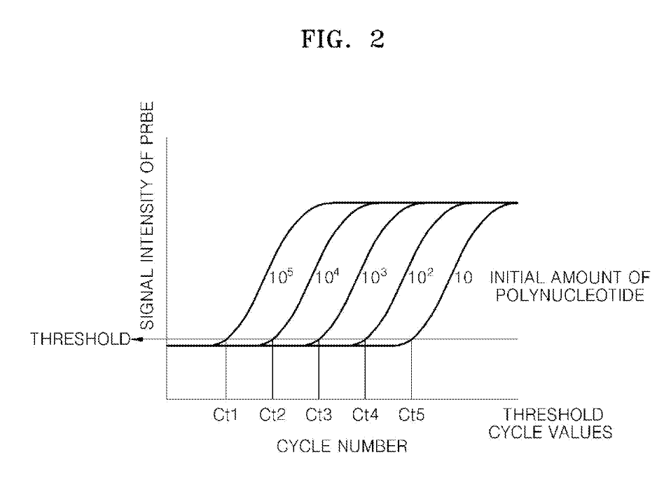

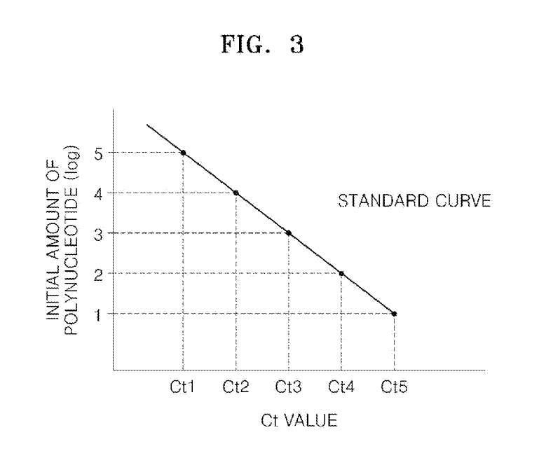

### DETAILED DESCRIPTION OF THE EXEMPLARY EMBODIMENTS

Hereinafter, the inventive concept will be described more specifically with reference to the following example. The following example is for illustrative purposes and is not intended to limit the scope of the inventive concept.

### Example 1

### Method of Outputting Standard Curve

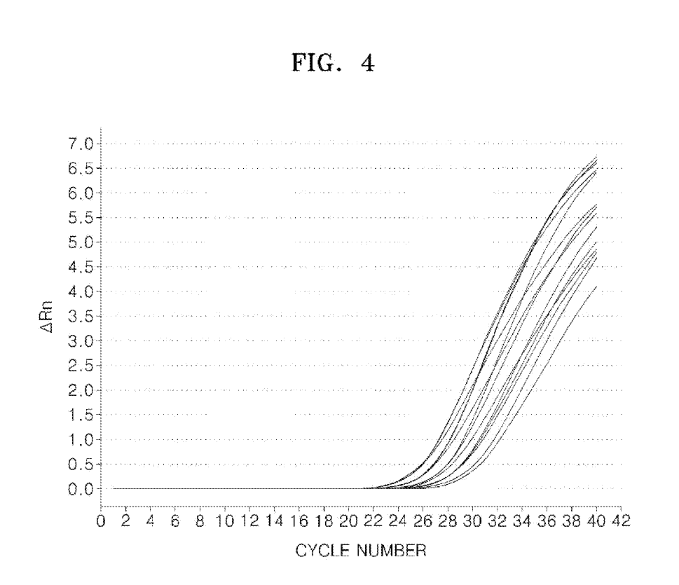

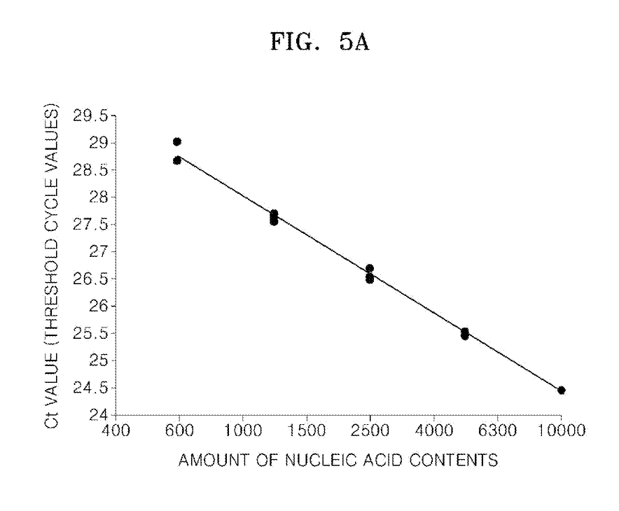

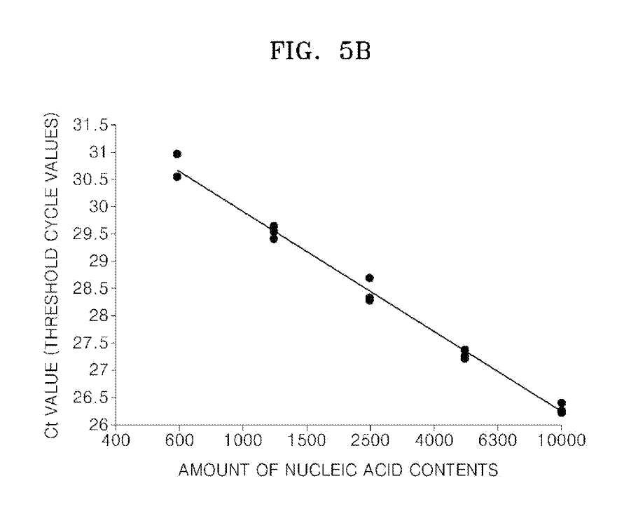

Initial nucleic acid contents of RNase P were 10000, 5000, 2500 1250, and 625 copies and three (3) same samples for each nucleic acid content were made. Real-time PCR was performed on a 15 samples by using an Applied Biosystems 7500 Real-Time PCR apparatus, thereby acquiring a data values of fluorescent signal intensities according to amplification cycles. Here, the number of amplification cycles performed was 40, and a used fluorescent probe was a TaqMan probe. Amplification profile curves based on the acquired data value of fluorescent signal intensity are shown in FIG. 4. In FIG. 4, a y-axis for a data value of a fluorescent signal intensity is indicated by ΔRn. 67 thresholds were selected with an interval of 0.1 in the range of about 0.1 to about 6.7. The selected thresholds were substituted for the amplification profile curves of FIG. 4, and Ct values according to the initial concentration are output. Two Ct values are selected ₁₅C₂ times in each threshold, and the selected Ct values are different each time. The two selected Ct values are used to output a standard curve, which is a primary straight line, and ₁₅C₂ standard curves are output for each threshold. An x-axis of the standard curve is a value of a log value of an amount of nucleic acid (copy numbers) (for convenience, values illustrated in an x-axis of FIG. 5 are the number of copies of nucleic acid), and a y-axis is a Ct value. In the standard curves, if an error between a standard curve and a Ct value was out of 0.5 threshold cycles or above, the Ct value was determined as an outlier. When an error between a standard curve and a Ct value was out of 0.5 threshold cycles or below, the Ct value was determined as an inlier. In each threshold, a standard curve including most inliers was selected. This indicates that the standard curve having the most number of Ct values within an error range of 0.5 threshold cycles or below is selected. Consequently, one standard curve was selected for each threshold, and then, a standard curve including the most inliers was selected from among the standard curves selected from each threshold. The selected standard curve included 15 samples as inliers, and the threshold was 0.2. The selected standard curve was based on two Ct values that are arbitrarily selected, and thus, a final standard curve was determined using 15 Ct values, which are output with the threshold of 0.2, by a simple linear regression analysis (FIG. 5A). A slope of the final standard curve was -3.5894 and an R² coefficient was 0.996. An R² coefficient of a standard curve calculated using an Applied Biosystems 7500 Real-Time PCR apparatus was 0.992 (FIG. 5B). The R² coefficient is a coefficient indicating whether the standard curve matches with a data value. As the R² coefficient is close to 1, the standard curve matches with threshold cycle data dots. Since the R² coefficient of the standard curve acquired in this example is higher than the standard curve acquired using the Applied Biosystems 7500 Real-Time PCR apparatus, the standard curve more matching with data can be acquired according to this example. Data in FIG. 5A is densely close to the standard curve compared with data in FIG. 5A so that it can be confirmed that a standard curve more matching with data is acquired in this example.

[Matlab Code Used to Execute Example 1]

function [CtList quantities threshold] =

ComputeCt(deltaRn,quantityList,standardIndex,nonStandardIndex)

%% Description
% ComputeCt finds the threshold cycle for each well
%
% INPUT deltaRn: normalized Reporter - baseline, [numCycles ×

numWells]

% INPUT quantityList : the quantity list of Standard samples [1 ×
% numStandards]
% INPUT standardIndex : the index list of Standard samples [ 1 ×
% numStandards]
% OUTPUT CtList: the list of the threshold cycle [1 × numWells]
% OUTPUT quantities : the list of the estimated quantities [1 ×

numWells]

% OUTPUT threshold : the estimated threshold [1 × 1]
if size(quantityList,2) ==1

quantityList = quantityList′;

end
if size(standardIndex,2) ==1

standardIndex = standardIndex′;

end
numWells = size(deltaRn,2);
numCycles = size(deltaRn,1);
numStandards = length(standardIndex);
CtList = zeros(1,numWells);
if nargin < 4

nonStandardIndex = setdiff(1:numWells,standardIndex);

end
%% Find the threshold cycles for each possible threshold
mx = max(deltaRn(:));
ThresholdList = 0.1:0.1:mx;
sz = length(ThresholdList);
CtMatrix = zeros(sz,numStandards);
for wellIndex = 1:numStandards,

CtMatrix(:,wellIndex) =

compCtMatrix(deltaRn,ThresholdList,standardIndex(wellIndex),
numCycles);

end
%% Find the best threshold using RANSAC
numIteration = 50;
% candidates: [score, slope, offset, the number of inliers]
candidates = zeros(sz,4);
% xs: quantity index
xs = log(quantityList)./log(10);
ErrThreshold = 0.5;
for index = 1:sz

ys = CtMatrix(index,:);
pts = sort(randi(numStandards,[numIteration 2]),2);
pts = pts(find(xs(pts(:,1)) ~= xs(pts(:,2))),:);
y2 = ys(pts(:,2));
y1 = ys(pts(:,1));
x2 = xs(pts(:,2));
x1 = xs(pts(:,1));
a = (y2−y1)./(x2−x1);
b = (y1.*x2 − y2.*x1)./(x2−x1);
A = repmat(a′,1,length(xs));
B = repmat(b′,1,length(xs));
Xs = repmat(xs,length(a),1);
Ys = A.*Xs + B;
%% find inliers and ninlier
err = abs(Ys − repmat(ys,length(a),1));
inliers = (err<ErrThreshold & Ys<= numCycles);
outliers = 1−inliers;
ninliers = sum(inliers,2);
fails = find(ninliers < max(ninliers));
%% compute scores
score = inliers.*err + outliers.*ErrThreshold;
score = mean(score,2);
score(fails) = Inf;
%%
[X I ] = min(score);
candidates(index,1) = score(I);
candidates(index,2) = a(I);
candidates(index,3) = b(I);
candidates(index,4) = ninliers(I);

end
%% Find the threshold with the most inliers
X = max(candidates(:,4));
selected = find(candidates(:,4)==X);
[X index] = min(candidates(selected,1));
index = selected(index);
%% Re-estimate the standard curve using all the inliers
CtList(standardIndex) = CtMatrix(index,:);
ys = CtMatrix(index,:);
if rem(length(xs),2) ==1

offset = mean(ys(find(median(xs)==xs)));

else

offset = mean(ys(find(median(xs(2:end))==xs)));

end
Y = ys−offset;
X = xs−median(xs);
slope = X′\Y′;
offset = mean(ys′−slope*xs′);
threshold = ThresholdList(index);
numNonStandards = length(nonStandardIndex);
for wellIndex = 1:numNonStandards,

CtList(nonStandardIndex(wellIndex)) =

compCtMatrix(deltaRn,threshold,nonStandardIndex(wellIndex),
numCycles);

end
quantities = 10.\hat{ }((CtList − offset)./slope);
quantities(standardIndex) = quantityList;
function Ct = compCtMatrix(deltaRn, Threshold, index, numCycles)
sz = length(Threshold);
x = 1:numCycles;
y = deltaRn(:,index)′;
xx = 1:0.01:numCycles;
yy = spline(x,y,xx);
yy = repmat(yy,sz,1);
xx = repmat(xx,sz,1);
Threshold = repmat(Threshold′,1,size(yy,2));
[X I ] = min(abs(yy−Threshold),[ ],2);
index = sub2ind(size(Threshold),1:sz,I′)′;
indexminus = sub2ind(size(Threshold),1:sz,I′−1)′;
Ct = xx(indexminus)+ ...

(Threshold(index)−yy(indexminus)).*(xx(index)−

xx(indexminus))./(yy(index)−yy(indexminus));

Ct = Ct′;

According to the above example, when a real-time PCR is performed, an accurate standard curve which reflects data from a plurality of amplification curves may be acquired, and thus, polynucleotide may be accurately quantified.

While the inventive concept has been particularly shown and described with reference to the above example, it will be understood by those of ordinary skill in the art that various changes in form and details may be made therein without departing from the spirit and scope of the inventive concept as defined by the following claims.

What is claimed is:

1. A method of acquiring a standard curve for quantifying polynucleotide by performing a real-time polynucleotide chain reaction (PCR), the method comprising:
(a) performing the real-time PCR for a plurality of samples having different initial polynucleotide concentrations, wherein the PCR is performed with respect to a plurality of amplification cycle numbers using detectable probes which provide a signal according to an amount of polynucleotide;
(b) acquiring a plurality of amplification profile curves with respect to signal intensity values provided by the probes according to the amplification cycle numbers;
(c) selecting one threshold from among the signal intensity values;
(d) calculating amplification cycle numbers corresponding to the selected thresholds from the plurality of amplification profile curves, and determining the calculated amplification cycle numbers as threshold cycle (Ct) values corresponding to each of the initial polynucleotide concentrations;
(e) selecting at least two Ct values among the Ct values determined in (d); and
(f) acquiring a standard curve from the selected Ct values.

2. The method of claim 1, further comprising:
(g) repeating (e) and (f) a plurality of times so that groups including at least two Ct values selected in (e) are different each time when (e) is repeated; and
(h) selecting a standard curve, which excludes outliers to the maximum and includes inliers to the maximum from among the Ct values determined in (d), from among the standard curves acquired in (g).

3. The method of claim 2, wherein, in (h), the selected standard curve has the most inliers among standard curves acquired by (g) and (h).

4. The method of claim 2, wherein, in (h), the selected standard curve excludes Ct values beyond a set range as outliers, and includes Ct values within the set range as inliers from among the Ct values determined in (d), from among the standard curves acquired in (g).

5. The method of claim 4, wherein the set range is from 0.1 to 1 cycle.

6. The method of claim 2, wherein, in (h), the selected standard curve has the smallest error between the selected standard curve and the Ct values determined in (d), among standard curves acquired by (g) and (h).

7. A method of acquiring a threshold for quantifying polynucleotide by performing a real-time polynucleotide chain reaction (PCR), the method comprising:
(a) performing the real-time PCR for a plurality of samples having different initial polynucleotide concentrations, wherein the PCR is performed with respect to a plurality of amplification cycle numbers using detectable probes which provide a signal according to an amount of polynucleotide;
(b) acquiring a plurality of amplification profile curves with respect to signal intensity values provided by the probes according to the amplification cycle numbers;
(c) selecting one threshold from among the signal intensity values;
(d) calculating amplification cycle numbers corresponding to the selected thresholds from the plurality of amplification profile curves, and determining the calculated amplification cycle numbers as threshold cycle (Ct) values corresponding to each of the initial polynucleotide concentrations;
(e) selecting at least two Ct values among the Ct values determined in (d); and
(f) acquiring a standard curve from the selected Ct values.
(g) repeating (e) and (f) a plurality of times so that groups including at least two Ct values selected in (e) are different each time when (e) is repeated; and
(h) selecting a standard curve, which excludes outliers to the maximum and includes inliers to the maximum from among the Ct values determined in (d), from among the standard curves acquired in (g)
(i) repeating (c), (d), (g), and (h) a plurality of times so that the threshold selected in (c) is different each time when (c) is repeated; and
(j) selecting a standard curve, which excludes the outliers to the maximum and includes the inliers to the maximum from among the Ct values determined in (d), from among the standard curves acquired in (h)
(k) selecting the threshold based on the selected standard curve determined in (j).

8. The method of claim 7, wherein, in (j), the selected standard curve has the most inliers among standard curves acquired by (i) and (j).

9. The method of claim 7, Wherein, in (j), the selected standard curve excludes Ct values beyond a set range as outliers, and includes Ct values within the set range as inliers from among the Ct values determined in (d), from among the standard curves acquired in (g).

10. The method of claim 9, wherein the set range is from 0.1 to 1 cycle.

11. The method of claim 7, wherein, in (j), the selected standard curve has the smallest error between the selected standard curve and the Ct values determined in (d), among standard curves acquired by (i) and (j).

12. A method of acquiring a threshold for quantifying polynucleotide by performing a real-time polynucleotide chain reaction (PCR), the method comprising:
(a1) performing the real-time PCR for a plurality of samples having different initial polynucleotide concentrations, wherein the PCR is performed with respect to a plurality of amplification cycle numbers using detectable probes which provide a signal according to an amount of polynucleotide;
(b1) acquiring a plurality of amplification profile curves with respect to signal intensity values provided by the probes according to the amplification cycle numbers;
(c1) selecting a plurality of different thresholds from among the signal intensity values;
(d1) calculating amplification cycle numbers corresponding to the selected thresholds from the plurality of amplification profile curves, and determining the calculated amplification cycle numbers as threshold cycle (Ct) values corresponding to each of the initial polynucleotide concentrations;
(e1) acquiring a plurality of standard curves for the plurality of selected thresholds based on the Ct values; and
(f1) selecting a standard curve, which excludes Ct values beyond a set range as outliers, and includes Ct values within the set range as inliers, from among the Ct values determined in (d1), from among the plurality of acquired standard curves.
(g1) selecting the threshold based on the selected standard curve determined in (f1).

13. The method of claim 12, wherein, in (f1), the selected standard curve has the most inliers among the standard curves acquired in (e1).

14. The method of claim 12, wherein, in (f1), the selected standard curve excludes Ct values beyond a set range as outliers, and includes Ct values within the set range as inliers, from among the Ct values determined in (d1), from among the plurality of acquired standard curves.

15. The method of claim 14, wherein the set range is from 0.1 to 1 cycle.

16. The method of claim 12, wherein, in (f1), the selected standard curve has the smallest error between the selected standard curve and the Ct values determined in (d1), among the standard curves acquired in (e1).

17. The method of claim 12, wherein in (c1), fixed sections in the signal intensity values are determined, and values that equally divide the fixed sections are selected as the plurality of different thresholds.

18. The method of claim 11, wherein in (e1), at least two of the Ct values determined in (d1) are selected, and the standard curves are acquired based on the selected Ct values.

19. The method of claim 18, wherein in (c1), fixed sections in the signal intensity values are determined, and values that equally divide the fixed sections are selected as the plurality of different thresholds.
